(19) United States
(12) Patent Application Publication
(10) Pub. No.: US 2018/0055857 A1
(43) Pub. Date: Mar. 1, 2018

(54) TREATMENT OF ACNE VULGARIS WITH SEQUENTIAL TOPICAL FORMULATIONS COMPRISING ANTIMICROBIAL ORGANIC ACID ANIONS IN ALKALINE PHASE

(71) Applicant: KANTIAN SCIENCES CORPORATION, Smithtown, NY (US)

(72) Inventor: Robert D. KROSS, Bellmore, NY (US)

(73) Assignee: Kantian Sciences Corporation, Smithtown, NY (US)

(21) Appl. No.: 15/558,882

(22) PCT Filed: Mar. 16, 2016
(86) PCT No.: PCT/US2016/022652

§ 371 Date: Sep. 16, 2017

Related U.S. Application Data
(60) Provisional application No. 62/133,507, filed on Mar. 16, 2015.

(51) Int. Cl. A61K 31/60; A61K 31/192; A61K 9/00; A61K 9/08

(52) U.S. Cl. CPC A61K 31/60; A61K 9/08; A61K 9/0014; A61K 31/192

(57) ABSTRACT

A method for treating skin conditions of a patient, including acne vulgaris, includes the steps of formulating an acidic aqueous solution having no less than a 0.1% concentration of mandelic acid and up to and including a 2.0% concentration of salicylic acid, formulating an alkaline aqueous solution having an alkaline nitrite salt as a first alkaline salt and a second alkaline salt of an acid selected from the anion of chlorous acid, fumaric acid, mandelic acid and a combination thereof. Thereafter, sequentially applying the acidic aqueous solution directly to the afflicted portion of the skin of the patient and, at least five minutes later, but no more than ten minutes later, applying the alkaline aqueous solution to that portion of the patient to which the acidic aqueous solution has already been applied.

### BACKGROUND OF THE INVENTION

### Technical Field of the Invention

The present invention relates to a multifunctional two-part liquid formulation in which each of the two parts is applied sequentially to the skin, with sufficient time between the two applications so that the keratolytic action of the first-applied, acidic phase is significantly underway before the residual acidity of that first-applied material on the skin subsequently converts certain acid anions in the second phase to the resultingly beneficial form, thereby providing supplemental and/or additional antimicrobial and/or keratolytic acids for the further alleviation of a variety of skin conditions, including acne vulgaris.

The invention provides for inter alia, in a preferred embodiment, an increased use level of salicylic acid as a keratolytc agent, up to the allowed level of 2% in most jurisdictions, by providing for the second-stage formation of such other keratolytic acids, as mandelic acid, which are “stored,” in their anion form, in the second-applied alkaline phase. In addition to the alkaline-nitrite salt, present in the second phase, which converts to the antimicrobial nitrous acid, upon combination with the first acid phase, the same approach can also be utilized by using an alkaline-chlorite salt, which similarly converts to the antimicrobial chlorous acid species upon combination with the first phase. And, in a related manner, such other anions of antimicrobial acids as e.g., fumaric, lactic and glycolic acids may be used to supplement, upon combination of the two phases, the antimicrobial action that can effectively destroy pathogenic microorganisms commonly found on the skin of subjects suffering from acne vulgaris and other skin conditions.

### Description of the Prior Art

The present invention is related to Kross, U.S. Pat. No. 8,932,650, issued Jan. 13, 2015, to the present inventor, and entitled “Multifunctional Topical Formulation for the Treatment of Acne Vulgaris and Other Skin Conditions.” The entirety of that patent is incorporated herein by reference.

The prior art described in Kross, U.S. Pat. No. 8,932,650 is of particular relevance when considering the inventive technology disclosed herein. As noted in the referenced U.S. patent, the areas in which that technology can find application include the broad range of topical skin infections, and disinfection, as well as conditions which involve both pathogenic microorganisms as well as physiologic dysfunction, and often a combination of both. In the latter category is the affliction termed acne, technically acne vulgaris, or commonly “acne.” Kross teaches an inventive composition, which is comprised of two aqueous solutions and/or gels, and/or creams, which are applied sequentially to the subject's skin, wherein the A and B phases are initially combined prior to topical application of the mixture to the skin, which includes two keratolytic agents, the β-hydroxy salicylic acid and an α-hydroxy acid; the latter in a preferred embodiment is mandelic acid. These acids are incorporated into Phase 1, the “acidifier,” and Phase 2 (as characterized in the '650 patent,) the “base,” which contains a nitrite salt, as the source (upon subsequent contact with residual acid Phase on the skin) of additional germicidal activity.

The '650 patent further teaches that the combination of the multiple acids in Phase 1 (salicylic+an α-hydroxy acid) with the nitrite-ion containing base, results in the formation of “a remarkably effective antimicrobial material, which surpasses even that of mandelic acid, by a wide margin. That highly-efficient antimicrobial is nitrous acid, which is formed, to a fractional (pH-dependent) degree, from the acidification of the nitrite ion by H⁺ ions in solution from the partial ionization of both salicylic and mandelic acids in the Acidifier phase” . . . the nitrous acid that is formed in the mixed matrix is at a balanced nitrous acid/nitrite ratio to ensure the desired efficacy and stability of the nitrous acid. The equilibrium this refers to is shown in the following relationship, where the pH (i.e. negative log of H⁺ concentration) determines the balance:

Na⁺NO₂⁻←±H⁺→HONO+Na⁺

### SUMMARY OF THE INVENTION

It is, therefore, an object of the present invention to provide a multi-functional composition directed to the treatment of acne vulgaris (acne) and other skin afflictions, where such composition allows for the maximum regulatory-approved level of salicylic acid of 2.0%.

It is a further object of the invention to achieve the maximum-permitted 2.0% salicylic acid level, and yet avoid any keratolytic-induced irritation by the inclusion of the preferred α-hydroxy mandelic acid in the first-applied acidic formulation in the inventive two-part composition, wherein there is at least a five-minute interval between skin application of the two phases comprising the claimed method, and where there is no ultimate loss of both the germicidal and keratolytic actions of lipophilic mandelic acid.

It is yet a further object of the invention to provide the mandelic acid as its anionic salt form in the formulation's base (alkaline phase), so that the subsequent application of the mandelate anion, as a component of the base which is applied to skin to which the acidic phase, containing inter alia salicylic acid, up to 2.0% of its composition, at least 5 minutes after its application, provides adequate H⁺ ion to convert the mandelate ion for keratolytic supplementation of that provided by that in the initially-applied salicylic acid phase.

And it is, yet, a further object of the invention that the mandelic acid provided by residual acidity in the first-applied acid phase, will also deposit an antimicrobial coating of mandelic acid on the skin, in addition to its function as a supplemental keratolytic agent.

It is an additional object of the present invention to supplement the base, as for the above-cited mandelate anion, with potentially germicidal acids, in their salt forms, those anion-to-acid transformations being nitrite-to-nitrous acid and chlorite-to-chlorous acid.

It is a further object of the present invention to supplement the base, as above, with an antimicrobial acid anion, where the acid form of the anion has a similar lipophilic characteristic to that of mandelic acid, such as fumaric acid.

And it is a further object of this invention to include the mandelic acid salt in the base phase with no limit, so the total combined level of mandelic acid, in both phases, represented by its level in the acid phase and the corresponding acid equivalent in the base phase, can exceed more than the 1% level of mandelic acid taught in the '650 patent in the acid phase.

The foregoing and related objects are accomplished by the topical application of an “A” and “B” pair, which are consistent with the above objects of the present invention. In that regard, the following Table provides representative two-part inventive compositions which are selectively-chosen and sequentially-applied, wherein the “A” phase is applied at least five and no more than ten minutes prior to skin application of the “B” phase. This selection is best described in the following Table of Options, which show an overview of the many inventive ways of improving upon that taught and suggested by Kross, U.S. Pat. No. 8,932,650.

REPRESENTATIVE OPTIONS FOR ACID AND BASE PHASES, AND COMMENTS
THERETO

| Acid Phase (A) | Base Phase (B) | Comments and |
|---|---|---|
| (excluding inactives) | (excluding inactives) | Rationale |
| 1) Sal. acid, up to 2.0% | Na⁺Nitrite 1.0+% | Note: no limit on mand. salt; it |
| Mand. acid <1.0% relative | Mand. salt to yield mand. acid in comb. with | can exceed equiv. of 1% |
| to sal. acid increase e.g., 0.5, | H⁺ of A | mand. acid shifted from A. |
| 0.1%, 0% | | Comb. can significantly |
| | | exceed orig.1% in A, for both |
| | | keratolytic and antimicrobial |
| | | actions. |
| 2) as in 1) supplemented with | Na⁺Nitrite 1.0+% | As above, except that the |
| acid source to trigger | Mand. salt to yield mand. acid in comb. with | chlorous acid formed upon |
| mandelate &/or chlorite anions, | H⁺ of A | contacting A is strictly a |
| to spare depletion of H⁺ from | Na⁺Chlorite % tbd to yield chlorous acid in | supplementary powerful |
| ionization of sal. acid. (e.g. | contact with H⁺ of A | antimicrobial. |
| H₃PO₄, KH₂PO₄) | | |
| 3) as in 1) &2) supplemented | Na⁺Nitrite 1.0+ | As above, except that the |
| with acid source to trigger | Mand. salt to yield mand. acid in comb.with | fumaric acid formed upon |
| mandelate + fumarate anions, | H⁺ of A | contact with skin residue of A |
| sparing depletion of H⁺ from | Fumarate salt → fumaric acid in contact | is a lipophilic anti-microbial |
| ionization of sal. acid. (e.g. H₃PO₄, | with part A | compound |
| KH₂PO₄) | | |
| 4) as in 1) supplemented with | No Na⁺Nitrite but otherwise same as Base of | As above, where the fumaric |
| acid source to trigger | 2) + | acid formed upon contact with |
| mandelate & chlorite anions, to | Mand. salt to yield mand. acid in comb. with | skin residue of A is a lipophilic |
| spare depletion of H⁺ from | H⁺ of A | antimicrobial compound |
| ionization of sal. acid. (e.g. | Na⁺Chlorite tbd to yield chlorous acid in | |
| H₃PO₄, KH₂PO₄) | contact w. A | |

As indicated, there are a variety of optional compositions to choose from, all within the purview of the possible inventive combinations of the “A” (acidic) and the “B” (base) phases, as in hereafter taught. Specifically, the procedure provides for applying to the subject's afflicted skin, ad seriatim, first the A (acidic) phase of the composition followed by, at least five minutes, but no more than ten minutes later, the subsequent topical application of the B (base) phase of a selected pair (i.e., an A and B set) of the inventive compositions.

### DETAILED DESCRIPTION OF THE PREFERRED EMBODIMENTS

The details regarding each of the Parts “A” and “B” compositions are shown to a greater degree in the following detailed description:
reduces the potential skin irritation induced by the part A keratolytic acids, as taught in my U.S '650 patent, i.e., salicylic and mandelic acids.
supplements the antimicrobial activity of the two part system resulting from the 2^{nd} phase combination of the B phase components.

The present invention is the result of a series of explorations of ways to: (a) minimize the potential for skin irritation; (b) increase the level of salicylic acid in the Part A phase, up to the United States Food and Drug Administration's (“FDA”) maximum approved 2% level of salicylic acid, as per its acne monograph; and (c) supplement the already high antimicrobial activity of the composition to further minimize the level of skin organisms, such as Propionibacterium acnes, the bacterium that can colonize the skin and hair follicles and which is recognized as the primary pathogenic agent associated with acne vulgaris. This included the search for other lipophilic and antimicrobial acids, and the anions thereof.

In regard to the other skin conditions or skin afflictions where microbial proliferation is a major problem, there are, for example, a number of bacterial skin infections and conditions, such as boils, folliculitis, carbuncles, furuncles, cellulitis, abscesses, impetigo, erysipelas, necrotizing fasciitis and Staphylococcus aureus infections, including MRSA, where a powerful topical antibacterial composition, as taught by this invention, could be decidedly beneficial. The same applies to common fungal infections, including athlete's foot, jock itch, ringworm, and such dermatophytoses as tinea versicolor.

### Initial Considerations:

There is a practical limitation to the level of use of both salicylic acid and another keratolytic acid (such as the mandelic acid in the preferred embodiment) in the two-part inventive composition. The FDA Monograph on the use of salicylic acid for the treatment of acne (U.S. Department of Health and Human Services, Food and Drug Administration: Part 333—Topical antimicrobial drug products for over-the-counter human use. Subpart D—Topical acne drug products. 21 C.F.R. 333.350) specifies in subsection (c) that salicylic acid may be used in topical products at a level in the “0.5 to 2 percent” range. Indeed this inventor has noted that every commercial product encountered for the treatment of acne vulgaris, and that contains salicylic acid, includes that material at the allowed maximum 2.0% level. However, in a commercial product based on the '650-patent, it has been virtually impossible to incorporate both salicylic acid, at the upper 2% level, in combination with the preferred mandelic acid (at an effective level) in a desired pH range. That is primarily because the mixture of those two acids borders on being “too irritating” to the skin of many subjects.

Accordingly, as a “compromise,” both keratolytic acids are included at the 1.0% level in the currently marketed product to achieve a significant degree of keratolysis provided by both acids with the concomitant benefit of mandelic acid's antimicrobial activity. However, it would certainly be beneficial to include the salicylic acid at the upper allowed 2% level. The present invention is a result of a series of discoveries and realizations developed during the course of investigating possible ways to achieve the goal of optimally balancing keratolysis and antimicrobial activity in the treatment of acne vulgaris and certain other skin conditions.

In considering ways of overcoming the problem of unwanted irritation associated with the keratolytic activity triggered by the two acids, in the first-applied acid phase, the inventor realized that it is possible to increase the level of salicylic acid in the acidic phase up to the 2% level by providing the preferred mandelic acid (and/or other keratolytic α-hydroxy acids, e.g., glycolic) in toto or in part, in its corresponding salt form, in the base phase; which already contains the nitrite ion. In this way, upon first applying the (acid) phase, the full FDA-allowed ≦2% level of salicylic acid can be utilized in the initial treatment phase without undue concern of irritation provoked by the additional presence of mandelic acid (as an example), and its keratolytic (and potentially irritating) activity. After a specific time interval of minimally five minutes, the subsequent application of the base phase can then be made. Acidity of the previously applied salicylic acid and either (a) smaller amounts and/or no amount of an α-hydroxy acid; and (b) if required, including supplemental non-irritating inorganic acids to the acid phase in order to readily transform both the nitrite and mandelate (or other α-hydroxy acid) ions to the desired corresponding acid form, at the desired pH.

This is achieved by formulating the acid phase so that the second stage application of the anion-containing phase to the residual acidity on the skin from the first acid phase application, will automatically create a system in the desired pH range, and thereby provide the enhanced antimicrobial action provided by the then-formed nitrous and mandelic acids and/or other keratolytic and antimicrobial acids. This is illustrated in the following set of equations, which depict the result of applying the base phase to the subject's skin, which firstly has residues of the salicylic acid phase. The new Phase A formulation can, if necessary, be augmented with an additional H⁺ supplying acid, to thereby spare the salicylic acid's H⁺ need for acidification of the additional anions “hiding” the Base phase. That H⁺ contributor can be an inexpensive, innocuous mineral acid (e.g., sodium dihydrogen phosphate NaH₂PO₄.H₂O), which, of course, would transform to the buffering mono-hydrogen, disodium phosphate (Na₂HPO₄), in whole or in part.

The nitrite/nitrous acid system Na⁺NO₂⁻+H⁺←→HONO+Na⁺

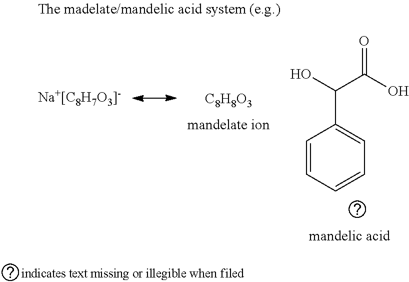

### Subsequent Considerations:

In exploring additional means for improving the technology taught in the '650 patent, this inventor realized that a chemical system which is remarkably analogous to that of the nitrous acid system can be utilized. That is the chlorous acid system, which shares nitrous acid's characteristic as an unstable acid which, as a result of its disproportionation at pHs in the ca. pH 3-4 range (optimal for this invention), creates highly effective antimicrobial systems (although non-keratolytic). Inventor Kross has several dozen US patents, as well as a number of patent applications, dealing with oxychlorine and oxynitrogen systems. Among their similarities is the fact that both nitrous acid and chlorous acid, as metastable species, both rapidly degrade (via disproportionation) as a function of pH, into species which, when considered together, retain the same total of balancing electronic configurations. For chlorous acid, formed from acidified chlorite ion, the overall reaction is as follows:

The chlorite/chlorous acid system Na⁺ClO⁻+H⁺←→HClO₂+Na⁺

As mentioned, the degree of conversion of the anionic to the acidic form is pH dependent. For the nitrite/nitrous acid system, that relationship, which covers the acidity range appropriate to formulations containing alkaline nitrite salts in the Base, in which the Acid phase residue on the skin after ≧5 minutes has pH values below from about 3.0 to about 3.5 or above, is as follows:

Percent of Nitrite as Nitrous Acid
Acid at Varying pH Values

| pH | % HONO | % NO₂⁻ |
|---|---|---|
| 2.6 | 83.3 | 16.7 |
| 2.8 | 76.0 | 24.0 |
| 3.0 | 66.7 | 33.3 |
| 3.3 | 50.0 | 50.0 |
| 3.5 | 38.8 | 61.2 |
| 4.0 | 16.6 | 63.4 |

For the analogous chlorite/chlorous acid system, when used as an alternate or supplemental antimicrobial to nitrite/nitrous acid, in combination with the keratolytic salicylic acid, with mandelate ion convertible to mandelic acid upon a ≧5-minute delayed contact with the acid phase remaining on the skin, the relationship over a similar pH range, is as follows:

Percent of Chlorite as Chlorous Acid at
Varying pH Values

| pH | % HClO₂ | % ClO₂⁻ |
|---|---|---|
| 2.3 | 35.0 | 68.0 |
| 2.6 | 18.1 | 81.9 |
| 2.8 | 12.3 | 87.7 |
| 3.0 | 8.5 | 91.5 |
| 3.5 | 2.4 | 97.6 |

As discussed above, the nitrite/nitrous acid and chlorite/chlorous acid metastable chemistries share the unique quality that, upon disproportionation, in that they form, inter alia, unstable free radical gases, which are NO (nitric oxide) and ClO₂ (chlorine dioxide), respectively. This then opens the possibility of including chlorite ion in the base phase, either as an alternate for the nitrite ion, or as a supplement thereto, so as to provide (if both are used) even more powerful antimicrobial activity against acne-related and/or other microbial species-associated with other skin conditions.

### Additional Considerations:

Other acid/anion pairs associated with keratolysis and/or supplemental activity against topical disease-associated conditions could be utilized. The α-hydroxy lactic acid, which possesses keratolytic activity, can be included judiciously as its lactate ion in the base phase, and the comparable α-hydroxy glycolic acid (with less antimicrobial action) can be utilized, in addition to other non-α-hydroxy acids, which have significant antimicrobial action. This includes acids such as fumaric acid,

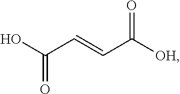

which, as for mandelic acid, also possesses high lipophilicity, thereby providing extended-cidal activity against skin organisms. Because of its lesser solubility in water, fumaric can be included as its single or double salt (i.e., the fumarate ion, mono- or divalent) in the base phase, for conversion to its active biocidal form upon application to the residual acid phase remaining on a patient's skin following application of the base phase, preferably, at a minimum of 5 minutes after the acid phase's application to the skin.

Summarizing the foregoing considerations, the present invention provides for:
a minimization of potential for skin irritation;
an increase in the level of salicylic acid in the Part A phase up to FDA's approved 2% acne monograph level; and,
supplementation of the already high antimicrobial activity of the composition which) include the search for other lipophilic and antimicrobial acids, and the anions thereof.

Technical advancements attendant the presently claimed invention include:

1. The ability to transfer up to 1% mandelic acid in the form of its anion to the base phase of the sequentially-applied acid-then-base formulations. This transfer can result in a reduction of potential irritation provoked by the combined salicylic and mandelic acids in the first applied acid phase. This allows the option of mandelic acid's partial transfer to the base (in its mandelate form), which indeed will allow the compounder to preserve some desired portion of the mandelic acid's initial antimicrobial activity, following the application of phase A, while facilitating the inclusion of more of the FDA's approved salicylic acid level (up to the allowed 2.0%) so as to reduce any corresponding skin irritation potential induced by the pair of keratolytic (i.e., literally (“skin dissolving”) acids. Implicit in this option is that, in effect this technique provides the compounder the ability to retain up to the full 1% of mandelic acid in phase A and have a supplementary amount of mandelic acid, in its salt form, in phase B at an equivalent level of ˜1%. In this manner, for commercial formulations, wherein there are minimal complaints of irritancy, the additional application of newly-formed mandelic acid, from phase B, to the residual H⁺ on the subject's skin, will provide an even greater amount of skin-adhering mandelic acid's long lasting antimicrobial activity.
2. The inclusion in the base phase of an additional nitrite-like anion, which upon acidification by the residual acid-phase on the skin, becomes a highly efficient, meta-stable germicidal acid, akin to a nitrite→nitrous acid conversion. That addition is the chlorous acid anion (i.e., chlorite), included in the base phase as an alkaline salt. The combination of each of these anions (nitrite and/or chlorite), partially becoming the corresponding nitrous and chlorous acids, at pH values in the acid range, approximating that of phase A's pHs of 3.5, provides even greater antimicrobial protection against harmful skin organisms, such as Propionibacterium acnes and such pathogenic Streptococcus species as S. pyogenes and such pathogenic Staphylococcus species as S. aureus, which causes deep-seated skin infections including furunculosis, that affects the skin's hair follicles.
3. Although fumaric has low water solubility, its very nature as a lipophilic acid, and possessing of antimicrobial activity, makes it an appropriate addition to the antimicrobial activity of the combined formulation when Part B is applied to the first-applied Part A. Hydrophobicity is important because the microbial cell wall normally contains lipid material which hydrophobic organic acids can interact with such lipid material and disrupt microbial activity. The anionic form of fumaric acid (e.g., sodium fumarate) converts to fumaric acid in the B+A mix on the skin, wherein the minimum water solubility of fumaric acid promotes its adherence to skin lipids. As an example of its antimicrobial activity, fumaric acid was found to be much more effective than lactic or acetic acids in the reduction of Listeria monocytogenes and E. coli O157:H7 in beef tissue briefly dipped in a 1% acid solution at 55° C.

In the application of this technology, it is often preferable to incorporate a thickening (gelling) agent to one or both parts of the two-part system, to add “body” to the composition. This allows for a greater deposition of the mixed material to skin surfaces and a greater duration of action. Gelling agents for use in the present invention include polysaccharides extracted from legume seeds, such as the galactomannans, including guar gum and locust bean (carob) gum. Other gelling agents include high molecular weight polyoxyalkylene cross-linked acrylic polymers as well as the highly preferred cellulosics such as hydroxymethyl cellulose, hydroxypropyl cellulose, hydroxyethyl cellulose, methyl cellulose, methylpropyl cellulose, among others, including high molecular weight polyethylene glycols, polyacrylamide and polyacrylamide sulfonates, and crosslinked polyvinylpyrrolidones, among others.

There are a variety of other microbially-associated skin disorders which can substantially benefit from the application of compositions incorporating the inventive powerful topical antibacterial technology. These conditions, as referenced above, include a host of other skin afflictions where microbial proliferation is a major problem. Examples in the category of bacterial skin infections and related conditions are boils, folliculitis, carbuncles, furuncles, cellulitis, abscesses, impetigo, and erysipelas. There is indeed a great need for an antimicrobial composition to treat diabetic foot ulcers, which result from deficient blood flow to peripheral tissues, such as in the feet of diabetes sufferers, where the necrotic tissue becomes a source of nutrients to invading bacteria. These often lead to foot amputation. There is the intriguing possibility, in such cases, that the nitric oxide. NO, which is generated from nitrous acid degradation can penetrate to underlying capillaries and stimulate their dilation, leading to enhanced blood flow, thereby helping to counter the ischemia resulting from diminished oxygen supply to the affected tissues. Similarly, the acidified chlorite system referenced above, which generates the proven antimicrobial chlorous acid composition, akin to the meta-stable nitrous acid system, optimally effective at pH's which are the focus of this inventive technology, will contribute similar protective and destructive activity against the variety of skin afflictions referenced above. Three of the antimicrobial acids utilized herein, mandelic, nitrous and chlorous acids, beside their demonstrated-cidal activity against the bacterium Propionibacterium acnes, will also be of potential benefit against such common bacterial and skin fungal infections as athlete's foot, jock itch, ringworm, furunculosis, keratosis pilaris, and the more than twenty species of the Candida yeast skin infections (e.g., candidiasis caused by Candida albicans). As indicated earlier, fumaric acid, which is created by Part A acid residue contact of the fumarate anion in Part B, has demonstrated antimicrobial activity in the reduction of a number of pathogens including Listeria monocytogenes and E. coli O157:H7, and being strongly lipophilic could well provide continued-cidal activity against a number of undesirable skin pathogens.

These compositions are preferably best applied in conjunction with a gel application medium, because of the ability of the gel to adhere to the skin. Any gelling agent or thickener that is non-toxic and non-reactive with the two phases may be used. For the acidic Part A, cellulose gels, particularly methyl and hydroxyethyl or hydroxypropyl cellulose gels, polyvinylsulfonic acid, polyamide and silica-based gels, are preferred. For the alkaline Part B, a number of beneficial skin agents, e.g. allantoin, and thickeners or gellant polymers, e.g. one of the “Carbopol” polymer derivatives, such as a member of the water soluble cross-linked polyacrylic acid copolymer family e.g., a member of the “Carbopol Ultrez” family may be included to promote skin adhesion of the composition. Other agents, which act as preservatives (e.g., members of the Euxyl family) and solubilizers and boosters (e.g., one of the Transcutols), add further integrity to the alkaline Part B. Preservatives may also be used when the gel form is employed. For example, sodium benzoate may be used as a preservative in the organic acid gel.

The present invention is illustrated by the following Examples. In these Examples the same preferred ingredients (other than the acids and their anionic counterparts) are used for all the formulations, while the nature and amounts of active agents (e.g., salicylic and mandelic in Part A, mandelate and/or nitrite and/or chlorite and/or fumarate anions in Part B) are included, at various levels in the A and B compositions. In these Examples, all parts and percentages therein, as well as the Specification and claims, are by weight, unless otherwise specified. The following Examples, which are non-limiting, further describe preferred embodiments within the scope of the present invention.

Many variations of these Examples are possible without departing from the spirit of the invention.

### EXAMPLES

### Example 1

This is a formulation for treating acne and other skin diseases, in which a significant portion (90%) of the mandelic acid taught in U.S. Pat. No. 8,932,650, and which was included in Part A to serve as a complementary keratolytic agent to salicylic acid, is included in Part B, in its anion form, i.e., as a mandelate salt. This reduction of mandelic acid's concentration in Part A allows for an increase of salicylic acid, to approach the FDA's allowed ≦2.0%, and thereby provides for less potential irritation from the combined keratolytic agents in Part A, for subjects sensitive to a 1%:1% salicylic:mandelic acid combination. The combination of both Parts A and B in this Example, as sequentially applied at a ≧5 minute separation, still preserves the extended antimicrobial activity of the lipophilic mandelic acid.

### Directions for Preparation of Both Parts A and B

### Part A:

Using less water (ca. 50%) in the initial mixture than needed to achieve the desired final volume, combine the various components in the following manner and numerical order indicated for each ingredient listed in the table that follows:
A) Mix the indicated amounts of 1) and 6) in a small container, and stir in 5). Into that liquid slowly disperse the required amount of 7) into the above liquids, ensuring uniform dispersion.
B) Fill a larger container of appropriate volume capacity to the intended batch size with ca. 90% of the water specified in 9), and stir in the required amount of 2).
C) With continued stirring add the required amounts of 4) then 3) until fully dissolved.
D) Add, with stirring, the A) mixture, and continue stirring until there is full and uniform dispersion of the Natrosol thickener (a trademark of Ashland Chemical for hydroxyethyl-cellulose (HEC), a nonionic, water-soluble polymer) throughout the bulk of the liquid.
At that point, and with continued stirring, add 8) the Blue #1 concentrate (a/k/a “Brillant Blue FCF”) until the solution is uniformly colored.
Now adjust the pH of the solution to ˜pH 3.2 dropwise with concentrated phosphoric acid (H₃PO₄), as needed. Finally, add sufficient deionized water to achieve the desired final volume, ensuring that the final pH remains at ˜3.2.

Part A Composition

| Component | Percentage |
|---|---|
| 1) Isopropyl alcohol (70%) | 35.70 |
| 2) Sodium benzoate | 0.04 |
| 3) Salicylic acid | 1.90 |
| 4) Mandelic acid | 0.10 |
| 5) Pluronic P103 | 0.10 |
| 6) Propylene glycol | 10.00 |
| 7) Natrosol 250 HHR | 1.30 |
| 8) Blue # 1 (0.1% sol'n) | 0.05 |
| 9) Deionized water | 50.81 |

Part B:

In this phase, it is more efficient to generate the required mandelate anion via its in situ preparation by dissolving the 1.00% mandelic acid in deionized water, using ˜90% of the intended final volume of Part B. Then add alkali (10% NaOH) dropwise sufficient to convert the mandelic acid to its corresponding salt (as verified by its pH≧8). Thereafter.
add the other ingredients in the amounts shown in the following table, sequentially, into a continuously stirring solution, first with the
sodium dodecylbenzene sulfonate, followed by the
Pluronic P103 (Pluronic P103 is a difunctional block copolymer surfactant terminating in primary hydroxyl groups. A nonionic surfactant that is 100% active and relatively nontoxic.) Then:
sodium nitrite; then disperse in the
Carbopol Ultrez 10 (Carbopol Ultrez 10 polymer is a cross-linked polyacrylic acid polymer that provides efficient theology modification with enhanced self-wetting for ease of use.), followed by the
Transcutol CG (Transcutol CG is ethoxydiglycol and is able to solubilize both hydrophilic and hydrophobic materials and is ideal for use in most fields of the cosmetic and personal care industry), then the
Euxyl PE 9010G (Euxyl PE 9010 is a liquid cosmetic preservative based on phenoxyethanol and ethylhexylglycerin), and lastly the
Allantoin (Allantoin is a chemical compound with formula C₄H₆N₄O₃, with the IUPAC name 5-ureidohydantoin or glyoxyldiureide.) Stir the final solution vigorously for at least 10 minutes, to ensure complete dispersion of all components.

Allantoin is a chemical compound with formula C₄H₆N₄O₃. It is also called 5-ureidohydantoin or glyoxyldiureide. Finally, adjust the pH to ˜8.5, dropwise with sufficient 10% NaOH or 10N HCl, then q.s. to the full volume with deionized water, and stir to uniformity.

Part B Composition

| Component | Percentage |
|---|---|
| Pluronic P103 | 0.10 |
| Sodium dodecylbenzenesulfonate | 0.10 |
| Sodium nitrite | 1.00 |
| Sodium mandelate | 1.03 |
| Carbopol Ultrez 10 | 1.00 |
| Allantoin | 0.10 |
| Euxyl PE 9010 | 1.00 |
| Transcutol CG | 1.00 |
| Deionized water | 94.70 |

The percentage of sodium mandelate is equivalent to 0.90% mandelic acid, upon contact with the residual skin acid(s) from Part A.

### Example 2

This is a formulation for treating acne and other skin diseases which retains the full 1.0% of the mandelic acid, as taught in Part A of U.S. Pat. No. 8,932,650, to serve as a complementary keratolytic agent to salicylic acid. Additionally the equivalent amount of 1.0% of mandelic acid, as its mandelate salt, is also included in Part B. The combination of both Parts A and B in Example 2, as sequentially applied at a ≧5-minute separation, provides supplemental antimicrobial activity of the lipophilic mandelic acid, a portion of which may well have been depleted by contact, and subsequent interaction with skin following application of Part A prior to the application of Part B.

### Directions for Preparation of Both Parts A and B

### Part A:

Using less water (ca. 50%) in the initial mixture than needed to achieve the desired final volume, combine the various components in the following manner and numerical order indicated for each ingredient in the table that follows: A) Mix the indicated amounts of 1) and 6) in a small container, and stir in 5). Into that liquid slowly disperse the required amount of 7) into the above pooled liquids, ensuring uniform dispersion.
B) Fill a larger container of appropriate volume capacity to the intended batch size with ca. 90% of the water specified in 9), and stir in the required amount of 2).
C) With continued stirring add the required amounts of 4) then 3) until fully dissolved.
D) Add, with stirring, the A) mixture, and continue stirring until there is full and uniform dispersion of the Natrosol thickener throughout the bulk of the liquid.
At that point, and with continued stirring, add 8) the Blue #1 concentrate until the solution is uniformly colored.
Now adjust the pH of the solution to ˜pH 3.2 dropwise with concentrated phosphoric acid (H₃PO₄) as needed.
Finally, add sufficient deionized water to achieve the desired final volume, ensuring that the final pH remains at ˜3.2.

Part A Composition

| Component | Percentage |
|---|---|
| 1) Isopropyl alcohol (70%) | 35.70 |
| 2) Sodium benzoate | 0.04 |
| 3) Salicylic acid | 1.00 |
| 4) Mandelic acid | 1.00 |
| 5) Pluronic P103 | 0.10 |
| 6) Propylene glycol | 10.00 |
| 7) Natrosol 250 HHR | 1.30 |
| 8) Blue # 1 (0.1% sol'n) | 0.05 |
| 9) Deionized water | 50.81 |

### Part B:

In this phase, it is more efficient to generate the required mandelate anion via its in situ preparation by dissolving the 1.00% mandelic acid in deionized water, using ˜90% of the intended final volume of Part B. Then add alkali (10% NaOH) dropwise sufficient to convert the mandelic acid to its corresponding salt (as verified by its pH≧8). Thereafter add the other ingredients in the amounts shown in the following table, sequentially, into a continuously stirring solution, first with:
sodium dodecylbenzene sulfonate, followed by the
Pluronic P103, followed by the
sodium nitrite; then disperse in the
Carbopol Ultrez 10, followed by the
Transcutol CG; then the
Euxyl PE 9010G, and lastly the
Allantoin.

Stir the final solution vigorously for at least 10 minutes, to ensure complete dispersion of all components. Finally, adjust the pH to ˜8.5, dropwise with sufficient 10% NaOH or 10N HCl, then q.s. to the full volume with deionized water, and stir to uniformity.

Part 3 Composition

| Component | Percentage |
|---|---|
| Pluronic P103 | 0.10 |
| Sodium dodecylbenzenesulfonate | 0.10 |
| Sodium nitrite | 1.00 |
| Sodium mandelate | 1.15 |
| Carbopol Ultrez 10 | 1.00 |
| Allantoin | 0.10 |
| Euxyl PE 9010 | 1.00 |
| Transcutol CG | 1.00 |
| Deionized water | 94.45 |

The percentage of sodium mandelate is equivalent to 1.00% mandelic acid, upon contact with the residual skin acid(s) of Part A.

### Example 3

This is a formulation for treating acne and other skin diseases which reduces the 1.0% mandelic acid to 0.10% while raising the level of salicylic acid to close to the ≦2.0% level allowed by the FDA for use in treating acne vulgaris. The Part B formulation, herein, contains the mandelic acid at a level convertible to 0.9% upon acidification by contact with the previously applied Part A. Additionally Part B contains the salts of both chlorous and nitrous acids (i.e., sodium chlorite and nitrite), so that the powerful metastable nitrous and chlorous germicides will be created when the Part B composition is in contact with the acidic skin residues of Part A are contacted at the ≧5 minute separation.

### Directions for Preparation of Both Parts A and B

### Part A:

Using less water (ca. 50%) in the initial mixture than needed to achieve the desired final volume, combine the various components in the following manner and numerical order indicated for each ingredient listed in the following table:
A) Mix the indicated amounts of 1) and 6) in a small container, and stir in 5). Into that liquid slowly disperse the required amount of 7) into the above pooled liquids, ensuring uniform dispersion.
B) Fill a larger container of appropriate volume capacity to the intended batch size with ca. 90% of the water specified in 9), and stir in the required amount of 2).
C) With continued stirring add the required amounts of 4) then 3) until fully dissolved.
D) Add, with stirring, the A) mixture, and continue stirring until there is full and uniform dispersion of the Natrosol thickener throughout the bulk of the liquid.
At that point, and with continued stirring, add 8) the Blue #1 concentrate until the solution is uniformly colored.
Now adjust the pH of the solution to ˜pH 3.2 dropwise with concentrated phosphoric acid (H₃PO₄) as needed.
Finally, add sufficient deionized water to achieve the desired final volume, ensuring that the final pH remains at ˜3.2.

Part A Composition

| Component | Percentage |
|---|---|
| 1) Isopropyl alcohol (70%) | 35.70 |
| 2) Sodium benzoate | 0.04 |
| 3) Salicylic acid | 1.90 |
| 4) Mandelic acid | 0.10 |
| 5) Pluronic P103 | 0.10 |
| 6) Propylene glycol | 10.00 |
| 7) Natrosol 250 HHR | 1.30 |
| 8) Blue # 1 (0.1% sol'n) | 0.05 |
| 9) Deionized water | 50.81 |

### Part B:

In this phase, it is more efficient to generate the required mandelate anion via its in situ preparation by dissolving the 1.00% mandelic acid in deionized water, using ˜90% of the intended final volume of Part B. Then add alkali (10% NaOH) dropwise sufficient to convert the mandelic acid to its corresponding salt (as verified by its pH≧18.5). Thereafter add the other ingredients in the amounts shown in the following table, sequentially, into a continuously stirring solution, first with:
sodium dodecylbenzene sulfonate, followed by the
Pluronic P103. Then followed by:
sodium nitrite; then disperse in the
Carbopol Ultrez 10, followed by the
Transcutol CG; then the
Euxyl PE 9010G, and lastly the
Allantoin.

Stir the final solution vigorously for at least 10 minutes, to ensure complete dispersion of all components.

Finally, adjust the pH to ˜8.5, dropwise with sufficient 10% NaOH or 10N HCl, then q.s. to the full volume with deionized water, and stir to uniformity.

Part B Composition

| Component | Percentage |
|---|---|
| Pluronic P103 | 0.10 |
| Sodium dodecylbenzenesulfonate | 0.10 |
| Sodium nitrite | 1.00 |
| Sodium chlorite | 1.30 |
| Sodium mandelate | 1.03 |
| Carbopol Ultrez 10 | 1.00 |
| Allantoin | 0.10 |
| Euxyl PE 9010 | 1.00 |
| Transcutol CO | 1.00 |
| Deionized water | 93.37 |

The percentage of sodium mandelate is equivalent to 0.90% mandelic acid, upon contact with the residual skin acid(s) of Part A.

### Example 4

This is a formulation for treating acne and other skin diseases which changes the 1.0% mandelic acid from Example 1 to 0.25% and contains 1.75% of salicylic acid, which is 87.5% of the maximum 2.0% level allowed by the FDA monograph for treating acne vulgaris. The Part B formulation contains the mandelic acid salt at a level equivalent of 1.0% mandelic acid upon contact with the previously applied Part A. Additionally Part B contains the salts of both chlorous and nitrous acids (i.e., sodium chlorite and nitrite), so that the powerful metastable nitrous and chlorous germicides will be created when the Part B composition is in contact with the skin residues of Part A are contacted at the ≧5 minute separation.

Directions for Preparation of Both Parts A and B

### Part A:

Using less water (ca. 50%) in the initial mixture than needed to achieve the desired final volume, combine the various components in the following manner and numerical order indicated for each ingredient in the table that follows:
A) Mix the indicated amounts of 1) and 6) in a small container, and stir in 5). Into that liquid slowly disperse the required amount of 7) into the above pooled liquids, ensuring uniform dispersion.
B) Fill a larger container of appropriate volume capacity to the intended batch size with ca. 90% of the water specified in 9), and stir in the required amount of 2).
C) With continued stirring, add the required amounts of 4) then 3) until fully dissolved.
D) Add, with stirring, the A) mixture, and continue stirring until there is full and uniform dispersion of the Natrosol thickener throughout the bulk of the liquid.
At that point, and with continued stirring, add 8) the Blue #1 concentrate until the solution is uniformly colored.
Now adjust the pH of the solution to ˜pH 3.2 dropwise with concentrated phosphoric acid (H₃PO₄) as needed. Finally, add sufficient deionized water to achieve the desired final volume, ensuring that the final pH remains at ˜3.2.

Part A Composition

| Component | Percentage |
|---|---|
| 1) Isopropyl alcohol (70%) | 35.70 |
| 2) Sodium benzoate | 0.04 |
| 3) Salicylic acid | 1.75 |
| 4) Mandelic acid | 0.25 |
| 5) Pluronic P103 | 0.10 |
| 6) Propylene glycol | 10.00 |
| 7) Natrosol 250 HHR | 1.30 |
| 8) Blue # 1 (0.1% sol'n) | 0.05 |
| 9) Deionized water | 50.81 |

### Part B:

In this phase, it is more efficient to generate the required mandelate anion via its in situ preparation by dissolving the 1.00% mandelic acid in deionized water, using ˜90% of the intended final volume of Part B. Then add alkali (10% NaOH) dropwise sufficient to convert the mandelic acid to its corresponding salt (as verified by its pH≧8.) Thereafter add the other ingredients in the amounts shown in the following table, sequentially, into a continuously stirring solution, first with:
sodium dodecylbenzenesulfonate, followed by the
Pluronic P103, then
sodium nitrite; and sodium chlorite. then disperse in the
Carbopol Ultrez 10, followed by the
Transcutol CG; then the
Euxyl PE 9010G, and, lastly, the
Allantoin.

Stir the final solution vigorously for at least 10 minutes, to ensure complete dispersion of all components. Finally, adjust the pH to ˜8.5, dropwise with sufficient 10% NaOH or 10N HCl, then q.s. to the full volume with deionized water, and stir to uniformity.

Part B Composition

Part B Composition

| Component | Percentage |
|---|---|
| Pluronic P103 | 0.10 |
| Sodium dodecylbenzenesulfonate | 0.10 |
| Sodium nitrite | 1.00 |
| Sodium chlorite | 1.30 |
| Sodium mandelate | 1.03 |
| Carbopol Ultrez 10 | 1.00 |
| Allantoin | 0.10 |
| Euxyl PE 9010 | 1.00 |
| Transcutol CO | 1.00 |
| Deionized water | 93.37 |

The percentage of sodium mandelate is equivalent to 1.00% mandelic acid, on contact with the residual skin acid(s) of Part A.

### Example 5

This is a formulation for treating acne and other skin diseases which reduces the Part A's 1.0% mandelic acid to 0.10%, while raising the level of salicylic acid to close to the ≦2.0% level allowed by the FDA for use in treating acne vulgaris. The Part B formulation, herein, contains the mandelic acid at a level convertible to 0.9% upon acidification by contact with the previously applied Part A. Additionally, Part B contains the salts of both fumaric and nitrous acids (i.e., fumarate and nitrite), so that the powerful metastable nitrous acid and lipophilic fumaric acid germicides will be created upon Part B's contact with the acidic skin residues of Part A following their ≧5-minute sequential application.

### Directions for Preparation of Both Parts A and B

Using less water (ca. 50%) in the initial mixture than needed to achieve the desired final volume, combine the various components in the following manner and numerical order indicated for each ingredient as indicated in the following table:
A) Mix the indicated amounts, serially, of 1), 6), then 5) in a small container. Into that liquid slowly disperse the required amount of 7) into the above pooled liquids, ensuring uniform dispersion.
B) Fill a larger container of appropriate volume capacity to the intended batch size with ca. 90% of the water specified in 9), and stir in the required amount of 2).
C) With continued stirring add the required amounts of 4) then 3) until fully dissolved.
D) Add, with stirring, the A) mixture, and continue stirring until there is full and uniform dispersion of the Natrosol thickener throughout the bulk of the liquid.
At that point, and continue stirring, add 8) until the solution is uniformly colored.
Now adjust the pH of the solution to ˜pH 3.2 dropwise with concentrated phosphoric acid (H₃PO₄) as needed. Finally, add sufficient deionized water to achieve the desired final volume, ensuring that the final pH remains at ˜3.2.

Part A Composition

| Component | Percentage |
|---|---|
| 1) Isopropyl alcohol (70%) | 35.70 |
| 2) Sodium benzoate | 0.04 |
| 3) Salicylic acid | 1.90 |
| 4) Mandelic acid | 0.10 |
| 5) Pluronic P103 | 0.10 |
| 6) Propylene glycol | 10.00 |
| 7) Natrosol 250 HHR | 1.30 |
| 8) Blue # 1 (0.1% sol'n) | 0.05 |
| 9) Deionized water | 50.81 |

### Part B:

In this phase, it is more efficient to generate the required mandelate anion via its in situ preparation by dissolving the 0.90% mandelic acid in deionized water, using ˜90% of the intended final volume of Part B. Then add alkali (10% NaOH) dropwise sufficient to convert the mandelic acid to its corresponding salt (as verified by its pH≧8.) Thereafter add the other ingredients in the amounts shown in the following table, sequentially, into a continuously stirring solution, first with:
two surfactants viz., sodium dodecylbenzenesulfonate 2) and Pluronic P103 1). Then weigh in the
sodium nitrite 3); then the disodium fumarate 4).
After complete solution is attained then disperse: Carbopol Ultrez 10 10) into the solution, followed by the
Transcutol CG 9); then the
Euxyl PE 9010G 8), and, lastly, the
Allantoin 7.

Stir the final solution vigorously for at least 10 minutes, to ensure complete dispersion of all components.

Finally, adjust the pH to ˜8.5, dropwise with sufficient 10% NaOH or 10N HCl, then q.s. to the full volume with deionized water, and stir to uniformity.

Part B Composition

| Component | Percentage |
|---|---|
| 1)- Pluronic P103 | 0.10 |
| 2)- Sodium dodecylbenzenesulfonate | 0.10 |
| 3)- Sodium nitrite | 1.00 |
| 4)- Disodium fumarate | 1.10* |
| 5)- Sodium mandelate | 1.03** |
| 6)- Carbopol Ultrez 10 | 1.00 |
| 7)- Allantoin | 0.10 |
| 8)- Euxyl PE 9010 | 1.00 |
| 9)- Transcutol CG | 1.00 |
| 10)- Deionized water | 93.57 |

*The disodium fumarate is equivalent to 0.80% fumaric acid, upon contact with the residual skin acid(s) of Part A.
**The sodium mandelate is equivalent to 0.90% mandelic acid, upon contact with the residual skin acid(s) of Part A.

While only several embodiments of the present invention have been shown and described, it will be obvious to those skilled in the art that many modifications may be made to the present invention without departing from the spirit and scope thereof.

1. A method for treating skin conditions of a patient, including acne vulgaris, comprising the steps of:
formulating an acidic aqueous solution having no less than a 0.1% concentration of mandelic acid and up to, and including a 2.0% concentration of salicylic acid;
formulating an alkaline aqueous solution having an alkaline nitrite salt as a first alkaline salt and a second alkaline salt of an acid selected from the group consisting of the anion of chlorous acid, the anion of fumaric acid, the anion of mandelic acid and a combination thereof; and,
sequentially applying said acidic aqueous solution directly to a portion of the skin of the patient and then applying said alkaline aqueous solution to the portion of the patient to which said acidic aqueous solution has already been applied, wherein upon contact with skin residues of said acidic aqueous solution, the skin residues are convertible to a germicidal acid, including said alkaline nitrite salt being converted to nitrous acid, whereby said germicidal acid supplements the activity of said acidic aqueous solution for treating skin conditions affecting the patient.

2. The method for treating skin conditions of a patient, including acne vulgaris, according to claim 1, wherein said step of sequentially applying said acidic aqueous solution directly to a portion of the skin of the patient and later applying said alkaline aqueous solution to the portion of the patient to which said acidic aqueous solution has already been applied is carried out by applying said alkaline aqueous solution to the portion of the patient approximately five minutes to approximately ten minutes later.

3. The method for treating skin conditions of a patient, including acne vulgaris, according to claim 1, wherein said second alkaline salt of an acid is an anion of mandelic acid at a concentration of 0.1% to 1.0%, based upon its molecular weight as a free acid.

4. The method for treating skin conditions of a patient, including acne vulgaris, according to claim 1, wherein said second alkaline salt of an acid is an anion of chlorous acid at a concentration of up to, and including, 1.0%, based upon its molecular weight as a free acid.

5. The method for treating skin conditions of a patient, including acne vulgaris, according to claim 1, wherein said second alkaline salt of an acid includes the anion of chlorous acid and the anion of mandelic acid.

6. The method for treating skin conditions of a patient, including acne vulgaris according to claim 1, wherein said acidic aqueous solution has a pH in the range of about 2.3 to about 4.0.

7. The method for treating skin conditions of a patient, including acne vulgaris, according to claim 6, wherein said acidic aqueous solution has a pH in the range of about 3.0 to about 3.5.

8. The method for treating skin conditions of a patient, including acne vulgaris, according to claim 7, wherein said acidic aqueous solution has a pH of about 3.2.

9. The method for treating skin conditions of a patient, including acne vulgaris, according to claim 1, wherein said aqueous solution has a pH of equal to or greater than 8.5.

10. The method for treating skin conditions of a patient, including acne vulgaris, according to claim 1, wherein said step of sequentially applying said acidic aqueous solution directly to a portion of the skin of the patient and, at least five minutes later, but no more than ten minutes later, applying said alkaline aqueous solution to that portion of the patient's skin to which said acidic aqueous solution has already been applied, is performed twice daily.

11. The method for treating skin conditions of a patient, including acne vulgaris, according to claim 1, further comprising the step of:
applying said alkaline aqueous solution that contains a gelling agent or thickener to the skin of the patient just before, or just after, applying said acidic aqueous solution, said gelling agent or thickener being non-toxic and non-reactive with each of said acidic aqueous solution and said basic aqueous solution.

12. The method for treating skin conditions of a patient, including acne vulgaris, according to claim 11, wherein said gelling agent or thickener is a member selected from the group consisting of a cellulose derivative, a polyvinylsulfonic acid, a polyamide polymer, a silica-based gellant and a combination thereof.

13. The method for treating skin conditions of a patient, including acne vulgaris, according to claim 12, wherein said cellulose derivative is a member selected from the group consisting of methyl cellulose, hydroxyethyl cellulose, hydroxypropyl cellulose and a combination thereof.

14. The method for treating skin conditions of a patient, including acne vulgaris, according to claim 1, further comprising the step of:
applying said alkaline aqueous solution that contains a gelling agent or thickener to the skin of a patient just before, or just after, applying said basic aqueous solution, said gelling agent or thickener being non-toxic and non-reactive with each of said acidic aqueous solutions and said basic aqueous solutions.

15. The method for treating skin conditions of a patient, including acne vulgaris, according to claim 1, wherein said gelling agent or thickener is a member selected from the group consisting of 5-ureidohydantoin, a water-soluble cross-linked polyacrylic acid polymer and a combination thereof.

16. A method for treating skin conditions of a patient, including acne vulgaris, comprising the steps of:
formulating an acidic aqueous solution having no less than a 0.1% concentration of mandelic acid, up to, and including a 2.0% concentration of salicylic acid, and up to, and including, a 1.0% concentration of a supplemental inorganic acidic salt;
formulating an alkaline aqueous solution having an alkaline salt of an acid including a nitrite salt and an additional acid salt selected from the group consisting of the anion of chlorous acid, the anion of fumaric acid, the anion of mandelic acid and a combination thereof; and,
sequentially applying said acidic aqueous solution directly to a portion of the skin of the patient and, at least five minutes later, but no more than ten minutes later, applying said alkaline aqueous solution to the portion of the patient to which said acidic aqueous solution has already been applied, wherein upon contact with skin residues of said acidic aqueous solution, the skin residues are convertible to a germicidal acid, whereby said germicidal acid supplements the activity of said acidic aqueous solution for treating skin conditions affecting the patient.
